(19) United States
(12) Patent Application Publication
(10) Pub. No.: US 2011/0195378 A1
(43) Pub. Date: Aug. 11, 2011

(54) Composite Bio-Ceramic Dental Implant and Fabricating Method Thereof

(75) Inventors: Sea-Fue Wang, Taipei (TW); Chung-Kuang Yang, Taipei (TW); Sheng-Yang Lee, Taipei (TW); Jen-Chang Yang, Taipei (TW); I-lin Ho, Taipei (TW)

(73) Assignee: NATIONAL TAIPEI UNIVERSITY OF TECHNOLOGY, Taipei (TW)

(21) Appl. No.: 12/777,646

(22) Filed: May 11, 2010

(30) Foreign Application Priority Data
Feb. 5, 2010 (TW) 99103511

(51) Int. Cl. A61C 13/00; A61K 6/02

(52) U.S. Cl. 433/167; 424/602; 424/489

(57) ABSTRACT

A composite bio-ceramic dental implant and fabricating method thereof are disclosed. The composite bio-ceramic is sintered at a temperature between 1000 and 1800° C. using the nearly inert bio-ceramic powder and the active bio-ceramic powder or the completely resorbable bio-ceramic powder. The bioactive bio-ceramic material is dispersed in the inert bio-ceramic material. Therefore, the composite bio-ceramic has enough mechanical strength and good bioactivity for dental implant.

### FIELD OF THE INVENTION

The present invention relates to a composite bioactive bio-ceramic dental implant and fabricating method thereof, particularly, to the dental implant with a bioactive bio-ceramic material dispersed in a nearly inert bio-ceramic material and fabricating method thereof.

### BACKGROUND OF THE INVENTION

Generally speaking, when choosing implant materials, the biocompatibility is firstly considered. For example, the dental implant should not cause blood coagulation and hemolysis reaction, or release any toxicant. Otherwise the tissues near the dental implant will have pathological changes. A dental implant with a smooth and stable surface releases fewer toxicants and irritants, but hardly binds the surrounding tissues. Thus, a fibrous capsule of 0.1˜10 μm is formed around the dental implant with a smooth and stable surface by the surrounding tissues. The fibrous capsule does not bind the dental implant, therefore after a long time it will have the outcomes that (1) fibrous capsules continues to thicken and blocks blood supply, so waste accumulates around the dental implant and inflammatory tissues are formed; (2) fibrous capsule calcification and sclerosis occur and causes local pain; and (3) the dental implant and the nearby tissues are damaged or hurt, or the dental implant loosens because of the unbalanced stress. In order to prevent the problems mentioned above, surface modifications such as etching, surface coating, etc, will be applied to the dental implant.

Metal is the most popular material in clinical treatment because of its good processability, simplicity for production, the ability to be deformed under stress, and the high melting point. Stainless steel, titanium alloy, and cobalt-chromium alloy are the widely-used metallic biological materials, wherein the titanium alloy is the most widely-used metals. Using metal as dental implant material will cause some problems. Using titanium as dental implant material needs another titanium material to bind the dental implant to an artificial tooth in the outer, where partial of the outer titanium material is exposed to the outside of the gum. The appearance is not decent, and the amount of spoiled bacteria increases between the outer titanium material and the gum. Furthermore, metal has lower biocompatibility. A metallic dental implant is connected with the gum by morphological connection and mechanical interlocking, thus the dental implant is easy to loosen. The metallic dental implant in a high electrolyte concentration body fluid will release metal ions. The concentration of the metal ions in the body will increase and harms human bodies.

Therefore, some of the dental implants use a nearly inert bio-ceramic material as the primary material. Ceramic materials are brittle, hard, strong in compression, and weak in shearing and tension. The surrounding tissues do not bind the dental implant using a nearly inert bio-ceramic material as the primary material. After a long time, the dental implant will have the fibrous capsules and problems mentioned above.

### SUMMARY OF THE INVENTION

The present invention discloses a composite bioactive bio-ceramic material comprising: a nearly inert bio-ceramic powder and a bioactive bio-ceramic powder. The bioactive bio-ceramic powder is well distributed in the nearly inert bio-ceramic powder via sintering. The nearly inert bio-ceramic material has lower mechanical strength. Adding bioactive bio-ceramic powder in a nearly inert bio-ceramic material can improve the mechanical strength, and resolve the problems of fibrous capsules.

The present invention discloses a composite bio-ceramic dental implant, comprising a nearly inert bio-ceramic material and a bioactive bio-ceramic material. The bioactive bio-ceramic material is well distributed in the nearly inert bio-ceramic material. Deciding the weight fraction of the nearly inert bio-ceramic material and the bioactive bio-ceramic material, phase continuity, phase connectivity and phase distribution can control the mechanical strength and biocompatibility of the dental implant.

The present invention discloses a method of fabricating dental implant, comprising: providing a composite bio-ceramic powder, comprising a nearly inert bio-ceramic powder and a bioactive bio-ceramic powder, and deciding the weight percentage of the bioactive bio-ceramic powder in the composite bio-ceramic powder; blending the composite bio-ceramic powder and a bonding agent; performing a forming technology to transfer the composite bio-ceramic powder and a bonding agent into a ceramic implant embryo; and performing a sintering process to transfer the ceramic implant embryo into a dental implant.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 shows a schematically diagram for fabricating dental implant;

FIG. 2A shows an X-ray diffraction pattern of pure zirconium oxide before sintering;

FIG. 2B shows an X-ray diffraction pattern of pure zirconium oxide after sintering at 1400° C. for 4 hours;

FIG. 3A shows an X-ray diffraction pattern of pure tricalcium phosphate before sintering;

FIG. 3B shows an X-ray diffraction pattern of pure tricalcium phosphate after sintering at 1400° C.; and

FIG. 4 shows an X-ray diffraction pattern of zirconium oxide 50 wt % and tricalcium phosphate after sintering at 1350° C. for 3 hours.

### DETAILED DESCRIPTION OF THE PREFERRED EMBODIMENT

The present invention provides composite bioactive bio-ceramic material can be used as a dental implant, knee implant, or orthopedic implant. Deciding the weight fraction of the nearly inert bio-ceramic material and the bioactive bio-ceramic material, phase continuity, phase connectivity and phase distribution can control the mechanical strength and biocompatibility of the bio-ceramic material.

The nearly inert bio-ceramic material is a stable material in a physiological environment. The nearly inert bio-ceramic material comprises one or the combination selected from a group of the following: zirconium oxide, aluminum oxide, and carbon base material, or comprises yttrium stabilized zirconia (YSZ) with a little transition metal or rare earth oxides. In a preferred embodiment, a little transition metal and rare earth oxides can improve the mechanical strength and toughness of the yttrium stabilized zirconia (YSZ) with biocompatibility.

The implant using pure nearly inert bio-ceramic material as the primary material will form fibrous capsules. Waste accumulates around the implant, and inflammatory tissues are formed. Therefore, the present invention provides the composite bioactive bio-ceramic material comprising the nearly inert bio-ceramic material and the bioactive bio-ceramic material. The bioactive bio-ceramic material is well dispersed in an inert bio-ceramic material via sintering, so that the composite bioactive bio-ceramic material can bind the surrounding tissues by chemical bonds.

The bioactive bio-ceramic powder comprises one or the combination selected from a group of the following: surface bioactive bio-ceramic material, and completely resorbable bio-ceramic material. When the surface bioactive bio-ceramic material is in a human body, a new material is formed on the surface between tissues and the surface bioactive bio-ceramic material. The new material will bind the surrounding tissues by chemical bonds for immobilization. The surface bioactive bio-ceramic material comprises one or the combination selected from a group of the following: hydroxyapatite, bioactive glass, and glass-ceramic.

The Hydroxylapatite, also called hydroxyapatite (HA, bond bonding, Ca₁₀(PO₄)₆(OH)₂), is the most widely-used bone material. The hydroxyapatite with good biocompatibility and Ca/P mole ratio of 1.67 is similar to the bone with Ca/P mole ratio of 1.6. The HA is used as a substrate for new bone cell to grow on it. The HA is applied in dental implant, artificial blood vessel, substrate, trachea, and laryngeal. The biocompatibility of an artificial joint can be improve by surface coating with the HA. The HA is weak in shearing and tension. Combining the HA and the nearly inert bio-ceramic material can solve the problem.

The HA can be divided into dense type and porous type. In a human body, the dense type HA is stable, but partial of the porous type HA will dissolve. While the porosity of the HA increases, the mechanical strength of the HA exponential decreases.

The HA can nucleate on the surface of a bioactive glass (SiO₂-P₂O₅—CaO—Na₂O). The glass-ceramic (SiO₂—CaO—Ca(PO₃)₂—Na₂O) comprises a micrite phase of the phosphorite.

The chemical compositions of the resorbable bio-ceramic material are similar to human tissues. The resorbable bio-ceramic material will dissolve and be absorbed in human bodies. The resorbable bio-ceramic material does not harm the human bodies. The resorbable bio-ceramic powder comprises one or the combination selected from a group of the following: tricalcium phosphate, calcium sulfate, and bio-ceramic. In a preferred embodiment, tricalcium phosphate is used as the resorbable bio-ceramic material. Tricalcium phosphate dissolves and disintegrates into many small particles. Then, tricalcium phosphate is absorbed.

In a preferred embodiment, the weight percentage of the bioactive bio-ceramic powder in said material ranges from 0.1% to 80%. The average particle diameter of the bioactive bio-ceramic powder ranges from 10 nm to 10 μm.

The second embodiment discloses a composite bio-ceramic dental implant, comprising a nearly inert bio-ceramic material and a bioactive bio-ceramic material. The bioactive bio-ceramic material is well distributed in the nearly inert bio-ceramic material.

Adding the bioactive bio-ceramic material with osteoinductive properties can reduce the osseointegration time.

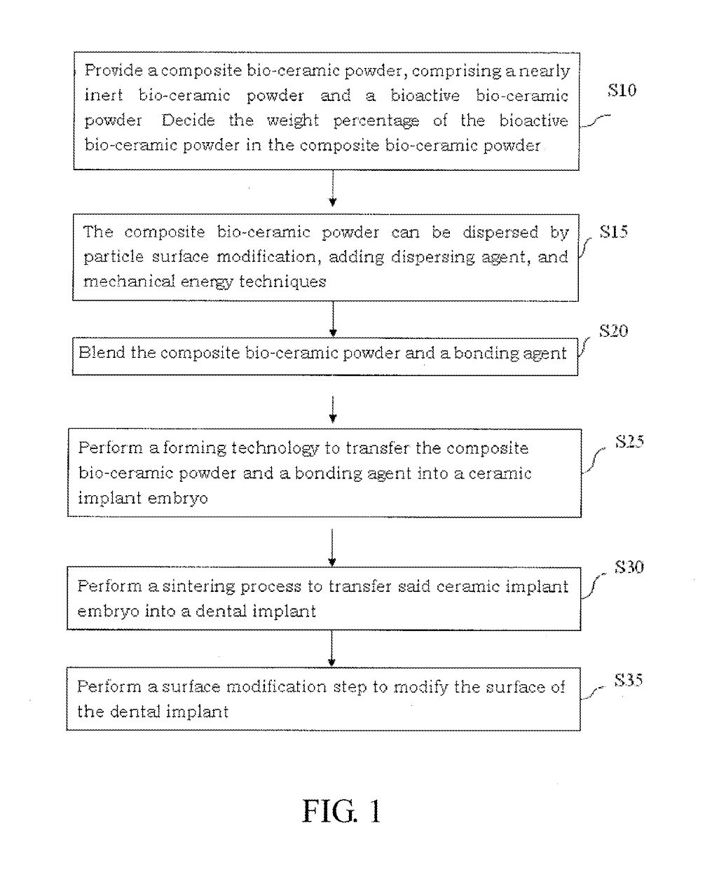

As shown in FIG. 1, the present invention discloses a method of fabricating dental implant, comprising:

S10: Provide a composite bio-ceramic powder, comprising a nearly inert bio-ceramic powder and a bioactive bio-ceramic powder. Decide the weight percentage of the bioactive bio-ceramic powder in the composite bio-ceramic powder. The nearly inert bio-ceramic powder comprises one or the combination selected from a group of the following: zirconium oxide, aluminum oxide, and carbon base material, or comprises yttrium stabilized zirconia (YSZ) with a little transition metal or rare earth oxides. In a preferred embodiment, the nearly inert bio-ceramic powder is the yttrium stabilized zirconia (YSZ) with a little transition metal and rare earth oxides

The bioactive bio-ceramic powder comprises one or the combination selected from a group of the following: surface bioactive bio-ceramic powder, and completely resorbable bio-ceramic powder. The surface bioactive bio-ceramic powder and the completely resorbable bio-ceramic powder are the materials mentioned above.

The weight percentage of the bioactive bio-ceramic powder in the material ranges from 0.1% to 80% and the average particle diameter of said bioactive bio-ceramic powder ranges from 10 nm to 10 μm. In a preferred embodiment, the completely resorbable bio-ceramic powder is tricalcium phosphate.

S15: The composite bio-ceramic powder can be dispersed by particle surface modification, adding dispersing agent, and mechanical energy techniques. The average particle diameter of the bioactive bio-ceramic powder ranges from 10 nm to 10 μm, so that the powder is easy to aggregate. The powder must be well-dispersed in blending, forming, and sintering. The dispersing agent comprises one or the combination selected from a group of the following: sodium carbonate, sodium silicate, sodium borate, tetrasodium pyrophosphate, sodium polymethacrylate, ammonium polyacrylate, sodium citrate, sodium succinate, sodium tartrate, sodium polysulfonate, and ammonium citrate.

S20: Blend the composite bio-ceramic powder and a bonding agent. The bonding agent comprises one or the combination selected from a group of the following: soluble silicate, soluble phosphates, soluble aluminates, organic silicates, Natural gums, polysaccharides, lignin extracts, refined alginate, cellulose ethers, polymerized alcohols, polymerized butyral, acrylic resins, glycols, waxes, kaolin, ball clay, bentonite, and microcrystalline cellulose.

S25: Perform a forming technology to transfer the composite bio-ceramic powder and a bonding agent into a ceramic implant embryo. In a preferred embodiment, the forming technology is an injection molding. Actually, forming technology can be the slip casting or compression molding.

S30: Perform a sintering process to transfer said ceramic implant embryo into a dental implant. Controlling the heating rate, cooling rate, sintering temperature, and sintering time of the sintering process yield a high quality microstructure of the dental implant. The sintering temperature of the sintering process ranges from 1000 to 1800° C. The sintering process does not cause any chemical reaction between the early inert bio-ceramic powder and the bioactive bio-ceramic powder, so that the dental implant has the advantages of the early inert bio-ceramic powder and the bioactive bio-ceramic powder.

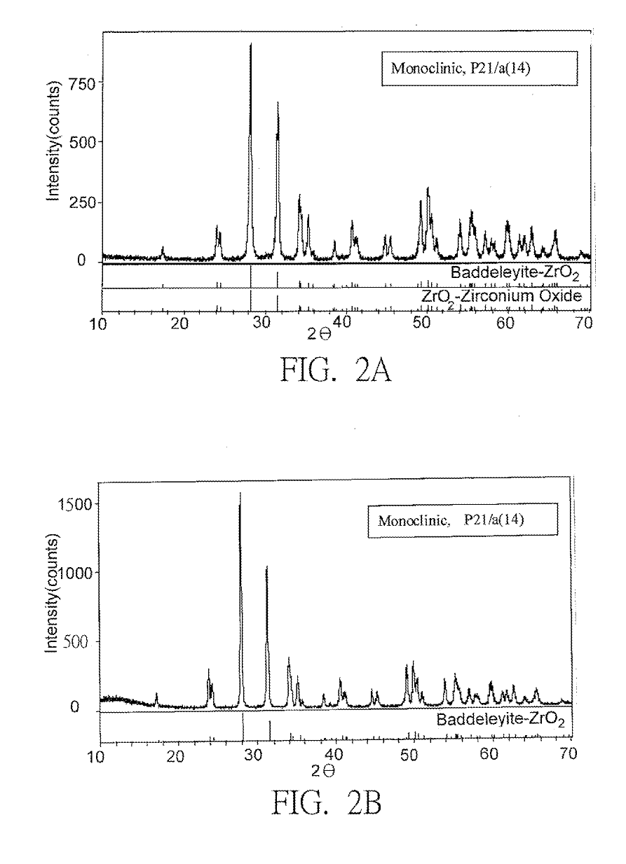

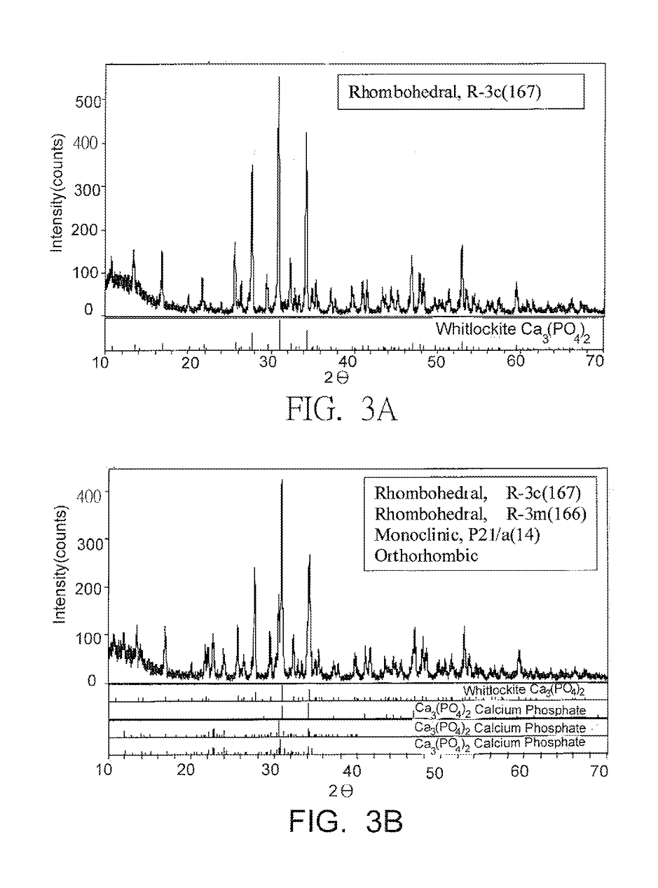

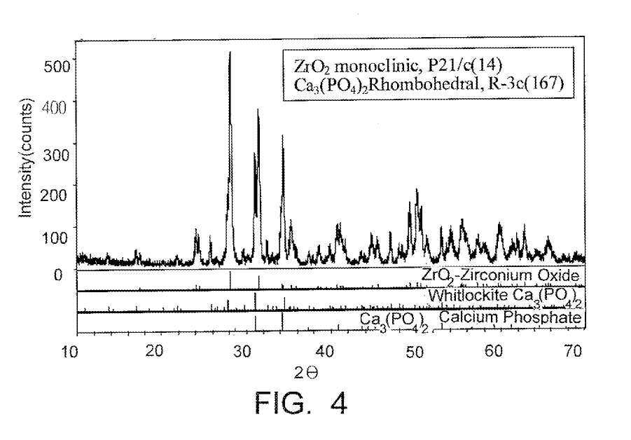

FIG. 2 to FIG. 4 are the experimental data of the preferred embodiment. FIG. 2A to FIG. 3A are the individual X-ray diffraction patterns of pure zirconium oxide and pure tricalcium phosphate before sintering. FIG. 2B and FIG. 3B are the individual X-ray diffraction patterns of pure zirconium oxide and pure tricalcium phosphate after sintering at 1400° C. for 4 hours. FIG. 4 is an X-ray diffraction pattern of zirconium oxide 50 wt % and tricalcium phosphate after sintering at 1350° C. for 3 hours.

FIG. 2A and FIG. 2B show that the pure zirconium oxide is monoclinic (P21/a (14)) before (and after) sintering. FIG. 3A shows that the pure tricalcium phosphate is rhombohedral before sintering, and FIG. 3B shows that the pure tricalcium phosphate forms different crystal structures after sintering. FIG. 4 shows that the primary diffraction peak of the tricalcium phosphate and zirconium oxide does not have chemical shift, so the two powders do not have any chemical reaction or chemical bond after sintering at 1350° C. for 3 hours. The two powders at the temperature between 1000° C. and 1800° C. do not have any chemical reaction.

S35: Perform a surface modification step to modify the surface of the dental implant for improving the accuracy of sizing and the biocompatibility of the dental implant. The surface modification step can chemically modify the surface or control the roughness. The surface modification step comprises electrochemistry method and chemical coating method. The surface modification step can help the dental implant to bind the tissue closely and improve the lifetime of the dental implant.

The present invention discloses the dental implant comprising the bioactive bio-ceramic material and the inert bio-ceramic material and has the advantages of the following:

(1) The pure nearly inert bio-ceramic material is brittle. Adding the bioactive bio-ceramic powder improves the mechanical strength of the dental implant.

(2) Adding the bioactive bio-ceramic powder makes the tissues bind the dental implant by chemical bond, or makes a lot of pores be formed on the surface of dental implant for the new bone cell to grow into the pores.

(3) The dental implant with the bioactive bio-ceramic material dispersed in the inert bio-ceramic material has the biocompatibility. The pure inert bio-ceramic material does not have the biocompatibility. The dental implant with the bioactive bio-ceramic material dispersed in the inert bio-ceramic material does not generate the fibrous capsules and the problems caused by the fibrous capsules.

(4) The material with the bioactive bio-ceramic material dispersed in the inert bio-ceramic material does not cause any reaction after sintering, so that the material keeps the original characters and has the advantages of both materials before sintering. Furthermore, deciding the weight fraction of the bioactive bio-ceramic material or the process parameters can control the properties of the material with the bioactive bio-ceramic material dispersed in the inert bio-ceramic material.

As is understood by a person skilled in the art, the foregoing preferred embodiment of the present invention is an illustration of the present invention rather than limiting thereon. It is intended to cover various modifications and similar arrangements included within the spirit and scope of the appended claims, the scope of which should be accorded the broadest interpretation so as to encompass all such modifications and similar structure.

What is claimed is:

1. A composite bioactive bio-ceramic material, said material comprising:
a nearly inert bio-ceramic powder and a bioactive bio-ceramic powder, wherein said bioactive bio-ceramic powder is well distributed over said nearly inert bio-ceramic powder via sintering.

2. The material according to claim 1, wherein said nearly inert bio-ceramic powder comprises one or the combination selected from a group of the following: zirconium oxide, aluminum oxide, and carbon base material, or comprises yttrium stabilized zirconia (YSZ) with a little transition metal or rare earth oxides.

3. The material according to claim 1, wherein said bioactive bio-ceramic powder comprises one or the combination selected from a group of the following: surface bioactive bio-ceramic powder, and completely resorbable bio-ceramic powder.

4. The material according to claim 3, wherein said surface bioactive bio-ceramic powder comprises one or the combination selected from a group of the following: hydroxyapatite, bioactive glass, and glass-ceramic, and said completely resorbable bio-ceramic powder comprises one or the combination selected from a group of the following: tricalcium phosphate, calcium sulfate, and bio-ceramic.

5. The material according to claim 4, wherein said bioactive glass is SiO₂-P₂O₅—CaO—Na₂O and said glass-ceramic is SiO₂—CaO—Ca(PO₃)₂—Na₂O.

6. The material according to claim 1, wherein the weight percentage of said bioactive bio-ceramic powder in said material ranges from 0.1% to 80%.

7. The material according to claim 1, wherein the average particle diameter of said bioactive bio-ceramic powder ranges from 10 nm to 10 μm.

8. A composite bio-ceramic dental implant, comprising:
a nearly inert bio-ceramic material and a bioactive bio-ceramic material, wherein said bioactive bio-ceramic material is well distributed in said nearly inert bio-ceramic material.

9. The dental implant according to claim 8, wherein said nearly inert bio-ceramic material comprises one or the combination selected from a group of the following: zirconium oxide, aluminum oxide, and carbon base material, or comprises yttrium stabilized zirconia (YSZ) with a little transition metal or rare earth oxides.

10. The dental implant according to claim 8, wherein said bioactive bio-ceramic material comprises one or the combination selected from a group of the following: surface bioactive bio-ceramic material, and completely resorbable bio-ceramic material.

11. The dental implant according to claim 10, wherein said surface bioactive bio-ceramic material comprises one or the combination selected from a group of the following: hydroxyapatite, bioactive glass, and glass-ceramic, and said completely resorbable bio-ceramic material comprises one or the combination selected from a group of the following: tricalcium phosphate, calcium sulfate, and bio-ceramic.

12. The dental implant according to claim 8, wherein the weight percentage of said bioactive bio-ceramic material in said dental implant ranges from 0.1% to 80%.

13. The dental implant according to claim 8, the average particle diameter of said bioactive bio-ceramic material ranges from 10 nm to 10 μm.

14. A method of fabricating dental implant, comprising:
providing a composite bio-ceramic powder, comprising a nearly inert bio-ceramic powder and a bioactive bio-ceramic powder, and deciding the weight percentage of said bioactive bio-ceramic powder in said composite bio-ceramic powder;
blending said composite bio-ceramic powder and a bonding agent;
performing a forming technology to transfer said composite bio-ceramic powder and a bonding agent into a ceramic implant embryo; and
performing a sintering process to transfer said ceramic implant embryo into a dental implant.

15. The method according to claim 14 further comprising a surface modification step to modify the surface of said dental implant.

16. The method according to claim 14 further comprising particle surface modification, adding dispersing agent, and mechanical energy techniques for dispersing said powders before blending said composite bio-ceramic powder and a bonding agent, wherein said dispersing agent comprises one or the combination selected from a group of the following: sodium carbonate, sodium silicate, sodium borate, tetrasodium pyrophosphate, sodium polymethacrylate, ammonium polyacrylate, sodium citrate, sodium succinate, sodium tartrate, sodium polysulfonate, and ammonium citrate.

17. The method according to claim 14, wherein controlling the heating rate, cooling rate, sintering temperature, and sintering time of said sintering process yield a high quality microstructure of said dental implant.

18. The method according to claim 14, wherein said bonding agent comprises one or the combination selected from a group of the following: soluble silicate, soluble phosphates, soluble aluminates, organic silicates, Natural gums, polysaccharides, lignin extracts, refined alginate, cellulose ethers, polymerized alcohols, polymerized butyral, acrylic resins, glycols, waxes, kaolin, ball clay, bentonite, and microcrystalline cellulose.

19. The method according to claim 14, wherein the sintering temperature of said sintering process ranges from 1000 to 1800° C.

20. The method according to claim 14, wherein said forming technology comprises one selected from a group of the following: injection molding, slip casting, and compression molding.

21. The method according to claim 14, wherein said nearly inert bio-ceramic powder comprises one or the combination selected from a group of the following: zirconium oxide, aluminum oxide, and carbon base material, or comprises yttrium stabilized zirconia (YSZ) with a little transition metal or rare earth oxides.

22. The method according to claim 14, wherein said bioactive bio-ceramic powder comprises one or the combination selected from a group of the following: surface bioactive bio-ceramic powder, and completely resorbable bio-ceramic powder.

23. The material according to claim 22, wherein said surface bioactive bio-ceramic powder comprises one or the combination selected from a group of the following: hydroxyapatite, bioactive glass, and glass-ceramic, and said completely resorbable bio-ceramic powder comprises one or the combination selected from a group of the following: tricalcium phosphate, calcium sulfate, and bio-ceramic.

24. The material according to claim 14, wherein the weight percentage of said bioactive bio-ceramic powder in said material ranges from 0.1% to 80%.

25. The material according to claim 14, wherein the average particle diameter of said bioactive bio-ceramic powder ranges from 10 nm to 10 μm.
